SEQUENCE LISTING (6 sequences)

SEQ ID NO: 1 - DNA, 23 bp, Artificial
  Synthetic primer
cagctctgacattctgattcgaa

SEQ ID NO: 2 - DNA, 20 bp, Artificial
  Synthetic primer
tgccacaagctgtccagtct

SEQ ID NO: 3 - DNA, 30 bp, Artificial
  Synthetic
aatcgacaggattccgattcctgaacagtg

SEQ ID NO: 4 - DNA, 21 bp, Artificial
  Synthetic primer
gtccctgccctttgtacacac

SEQ ID NO: 5 - DNA, 19 bp, Artificial
  Synthetic primer
cgatccgagggcctcacta

SEQ ID NO: 6 - DNA, 18 bp, Artificial
  Synthetic primer
ctgcaagcatataataca

(19) United States
(12) Patent Application Publication
(10) Pub. No.: US 2010/0120851 A1
(43) Pub. Date: May 13, 2010

(54) PRENYLTRANSFERASE INHIBITORS FOR OCULAR HYPERTENSION CONTROL AND THE TREATMENT OF GLAUCOMA

(75) Inventors: Allan R. SHEPARD, Fort Worth, TX (US); Debra L. FLEENOR, Crowley, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/614,104

(22) Filed: Nov. 6, 2009

Related U.S. Application Data
(63) Continuation of application No. 11/692,316, filed on Mar. 28, 2007, now abandoned.
(60) Provisional application No. 60/787,971, filed on Mar. 31, 2006.

(51) Int. Cl. A61K 31/197; A61P 17/02; A61K 31/4164; A61K 31/4709

(52) U.S. Cl. 514/312; 514/562; 514/539; 514/399

(57) ABSTRACT

The invention concerns in one embodiment a method of treating glaucoma or elevated intraocular pressure comprising administering a pharmaceutically effective amount of a composition comprising at least one prenyltransferase inhibitor. In another embodiment, the invention concerns a composition for the treatment of elevated intraocular pressure and glaucoma comprising a pharmaceutically effective amount of a prenyltransferase inhibitor.

### CROSS-REFERENCE TO RELATED APPLICATION

This application is a Continuation (CON) of co-pending U.S. application Ser. No. 11/692,316, filed Mar. 28, 2007, priority of which is claimed under 35 U.S.C. §120, the contents of which are incorporated herein by reference. This application also claims priority under 35 U.S.C. §119 to U.S. Provisional Patent Application No. 60/787,971, filed Mar. 31, 2006, the contents of which are incorporated herein by reference.

### TECHNICAL FIELD OF THE INVENTION

The present invention is generally related to treatments for ocular hypertension and glaucoma, and more specifically related to prenyltransferases inhibitors for the treatment of ocular hypertension and glaucoma.

### BACKGROUND OF THE INVENTION

The disease state referred to as glaucoma is characterized by a permanent loss of visual function due to irreversible damage to the optic nerve. The several morphologically or functionally distinct types of glaucoma are typically characterized by elevated intraocular pressure (IOP), which is considered to be causally related to the pathological course of the disease. Ocular hypertension is a condition wherein intraocular pressure is elevated, but no apparent loss of visual function has occurred; such patients are considered to be at high risk for the eventual development of the visual loss associated with glaucoma. If glaucoma or ocular hypertension is detected early and treated promptly with medications that effectively reduce elevated intraocular pressure, loss of visual function or the progressive deterioration thereof can generally be ameliorated. Also, some patients with glaucomatous field loss have relatively low intraocular pressure. These so-called normotension or low tension glaucoma patients can also benefit from agents that lower and/or control IOP.

Drug therapies that have proven to be effective for the reduction of intraocular pressure include both agents that decrease aqueous humor production and agents that increase the outflow facility. Such therapies are in general administered by one of two possible routes, topically (direct application to the eye) or orally. However, pharmaceutical ocular anti-hypertension approaches have exhibited various undesirable side effects. For example, miotics such as pilocarpine can cause blurring of vision, headaches, and other negative visual side effects. Systemically administered carbonic anhydrase inhibitors can also cause nausea, dyspepsia, fatigue, and metabolic acidosis. Certain prostaglandins cause hyperemia, ocular itching, and darkening of eyelashes and periorbital skin. Such negative side-effects may lead to decreased patient compliance or to termination of therapy such that normal vision continues to deteriorate. Additionally, there are individuals who simply do not respond well when treated with certain existing glaucoma therapies. There is, therefore, a need for other therapeutic agents for the treatment of glaucoma and ocular hypertension.

Prenyltransferases are part of the isoprenoid biosynthetic pathway which includes cholesterol synthesis and the formation of mevalonate. Downstream metabolites of mevalonate such as geranylgeranyl pyrophosphate (GGPP) and farnesyl pyrophosphate (FPP) are used for post-translational processing of proteins. During such processing, the prenyltransferases FTase and GGTase transfer farnesyl (C15) or geranylgeranyl (C20) lipid anchors to protein cysteine residues in the C-terminal amino acid motif CAAX. Processed proteins such as Ras, Rab, and Rho may be involved in cell growth, cell signaling, and apoptosis (Doll, et al., Curr Opin Drug Discov Devel., Vol. 7(4):478-486, 2004). Particularly, Rho-dependent changes in cellular actin cytoskeletons can result in alterations in cell shape, contractility and motility, perhaps involving ocular tissue (Rao et al., IOVS, Vol. 42:1029, 2001; Rao et al., Exp Eye Res, Vol. 80:197-206, 2005; Cellini et al., Ophth Res, Vol. 37:43-49, 2005). The role of prenyltransferases in cancerous disease states is actively being explored in the art.

Agents such as connective tissue growth factor (CTGF) and Plasminogen Activator Inhibitor-1 (PAI-1) produced by trabecular meshwork cells may be elevated during conditions of elevated IOP. Kirwan et al., Glia., Vol. 52(4):309-24, 2005; Liton et al., J Cell Physiol., Vol. 205(3):364-71, 2005; Esson et al., Invest Ophthalmol Vis Sci., Vol. 45(2):485-91, 2004; Daniels et al., Am J Pathol., Vol. 163(5):2043-52, 2003; Liang et al., J Biol Chem., Vol. 278(29):27267-77, 2003; Ho, et al., Br. J. Ophthalmol., Vol. 89:169-173, 2005,. Such agents may therefore contribute to the pathogenesis of glaucoma.

### BRIEF SUMMARY OF THE INVENTION

The invention relates to the treatment of glaucoma and ocular hypertension using inhibitors of the prenyltransferases geranylgeranyltransferase (GGTase) and farnesyltransferase (FTase). Embodiments of the present invention recognize that

GGTase and/or FTase inhibitors may alter aqueous humor outflow and prove beneficial for treatment of ocular hypertension and glaucoma. Delivery of these inhibitors occurs via topical ocular, intracameral, intravitreal, subretinal, or transcleral administration in preferred embodiments.

Certain compounds contemplated by the invention may possess both GGTase and FTase inhibitory activity and may be administered singly or in a composition. In other embodiments, separate GGTase inhibitory and FTase inhibitory compounds are administered, either together in the same composition or separately by themselves or in different compositions.

A further feature of the invention is to provide a method of treating or preventing glaucoma which provides for a significant reduction in the production of connective tissue growth factor (CTGF) and Plasminogen Activator Inhibitor-1 (PAI-1) by trabecular meshwork cells.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1 is a graph of the effects of a geranylgeranyltransferase inhibitor on basal and TGFβ2-induced CTGF gene expression in TM cell lines;

FIG. 2 is a graph of the effects of a farnesyltransferase inhibitor on basal and TGFβ2-induced CTGF gene expression in TM cell lines;

FIG. 3 is a graph of the effects of a geranylgeranyltransferase inhibitor and a farnesyltransferase inhibitor on basal and TGFβ2-induced PAI-1 gene expression in TM cell lines; and

FIG. 4 shows graphs presenting cytotoxicity effects of a geranylgeranyltransferase inhibitor and a farnesyltransferase inhibitor.

### DETAILED DESCRIPTION OF THE INVENTION

The present invention relates in several embodiments to GGTase and FTase inhibitors for the treatment of ocular hypertension and glaucoma. Other embodiments comprise methods for treating ocular hypertension and glaucoma by administering such GGTase and FTase inhibitory compounds. Administration of the GGTase/FTase inhibitors according to embodiments of the present invention may allow the inhibitors to reach the appropriate target tissue, such as the trabecular meshwork, at therapeutic levels thereby alleviating and preventing further ocular damage resulting from glaucoma.

GGTase inhibitors used in embodiments of the present invention comprise, among others, the GGTase inhibitory compounds listed in U.S. Pat. Nos. 6,693,123; 6,627,610; 6,210,095; 6,221,865; 6,204,293; 5,965,539; and 5,789,558; herein incorporated by reference.

FTase inhibitors used in embodiments of the present invention comprise, among others, the FTase inhibitory compounds listed in U.S. Pat. Nos. 6,693,123; 6,627,610; 6,310,095; 6,221,865; 6,218,375; 6,204,293; 6,083,985; 6,083,917, 6,011,175; 5,856,310; and 5,834,434; herein incorporated by reference. Additional FTase inhibitors used in embodiments of the present invention are FTI-276, FTI-277, L-739,749, L-739,750, L-745,631, RPR-130401, BMS-193269, BMS-184878, SCH-66336, BZA-2B, BZA-5B, R-115777, B956, B1086, and Farnesylmethylhydroxyphosphinyl methyl phosphonic acid (Sebti et al., Exp Opin Invest Drugs, Vol. 9(12):2767-2782, 2000; Sebti, The Oncologist, Vol. 8(Supp 3):30-38, 2003).

Certain embodiments of the present invention comprise compounds with both GGTase and FTase inhibitory activity and are generally peptidomimetic inhibitors based on the CAAX motif. Examples of such compounds include, but are not limited to, C—V—I-M, C—V-L-L, FTI-276, FTI-277, GGTI-297, GGTI-298, FTI-2148, FTI-2153, GGTI-2154, GGTI-2166, R115777, SCH66336, HFPA (Sebti et al., Exp Opin Invest Drugs, Vol. 9(12):2767-2782, 2000); Sebti, The Oncologist, Vol. 8(Supp 3):30-38, 2003). Modifications of the imidazole-methyl diaryl ether structure have been shown to have dual FTase and GGTase inhibitory activity (FTase IC₅₀=2.9 nM, GGTase IC₅₀=7.1 nM). Several of these compounds are shown below, along with compounds having GGTase-specific activity (GGTI-286 and GGTI-298):

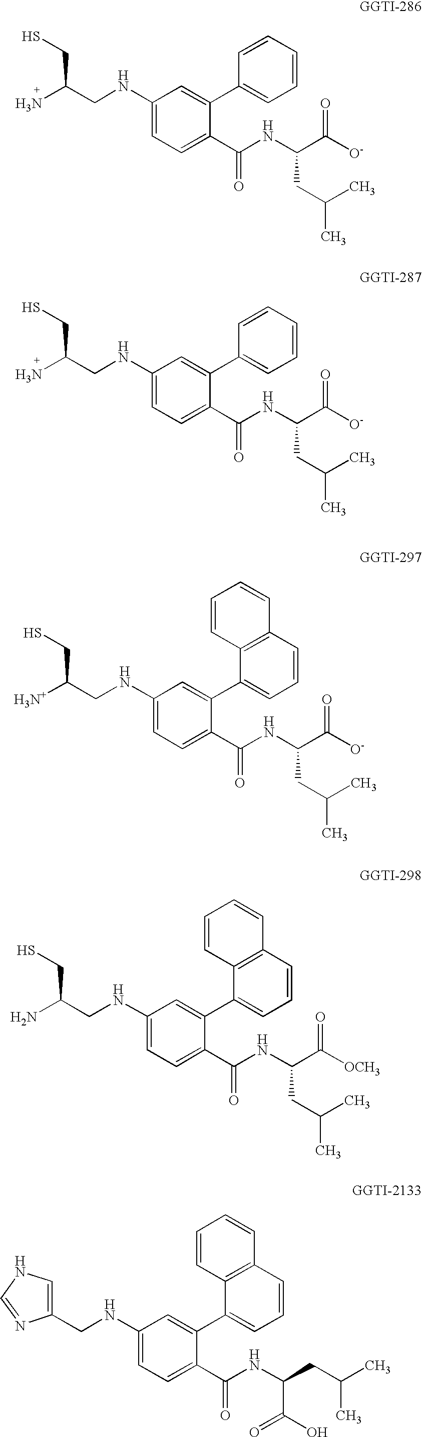

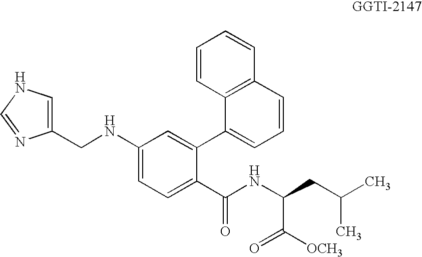

Inhibition constants are available for the above, commercially available compounds and are presented in Table 1 below. These compounds can also be synthesized using techniques known to those of skill in the art.

TABLE 1
Inhibition Constants for Selected Prenyltransferase Inhibitors

| Compound ID | Source/Cat# | GGTase IC50 | Ftase IC50 |
|---|---|---|---|
| Ftase Inhibitor I | Calbiochem #344510 | 790 nM | 21 nM |
| Ftase Inhibitor II | Calbiochem #344512 | | 50 nM |
| Ftase Inhibitor III | Calbiochem #344514 | | 12 nM |
| FTI-276 | Calbiochem #344550 | 50 nM | 500 pM |
| FTI-277 | Calbiochem #344555 | | 100 nM |
| GGTI-286 | Calbiochem #345878 | 2 uM | |
| GGTI-287 | Calbiochem #345880 | 5 nM | 25 nM |
| GGTI-297 | Calbiochem #345882 | 50 nM | 200 nM |
| GGTI-298 | Calbiochem #345883 | 3 uM | |
| GGTI-2133 | Calbiochem #345884 | 38 nM | 5.4 uM |
| GGTI-2147 | Calbiochem #345885 | 500 nM | 30 uM |

It is recognized that compounds disclosed herein can contain one or more chiral centers. This invention contemplates all enantiomers, diastereomers, and mixtures of compounds disclosed herein. Furthermore, certain embodiments of the present invention comprise pharmaceutically acceptable salts of disclosed compounds. Pharmaceutically acceptable salts comprise, but are not limited to, soluble or dispersible forms of compounds that are suitable for treatment of disease without undue undesirable effects such as allergic reactions or toxicity. Representative pharmaceutically acceptable salts include, but are not limited to, acid addition salts such as acetate, citrate, benzoate, lactate, or phosphate and basic addition salts such as lithium, sodium, potassium, or aluminum.

It is important to recognize that a substituent may be present either singly or multiply when incorporated into the indicated structural unit. For example, the substituent halogen, which means fluorine, chlorine, bromine, or iodine, would indicate that the unit to which it is attached may be substituted with one or more halogen atoms, which may be the same or different.

### Modes of Delivery

The GGTase and FTase inhibitory compounds of the present invention can be incorporated into various types of ophthalmic formulations for delivery. The compounds may be delivered directly to the eye (for example: topical ocular drops or ointments; slow release devices such as pharmaceutical drug delivery sponges implanted in the cul-de-sac or implanted adjacent to the sclera or within the eye; periocular, conjunctival, sub-tenons, intracameral, intravitreal, or intracanalicular injections) or systemically (for example: orally, intravenous, subcutaneous or intramuscular injections; parenterally, dermal or nasal delivery) using techniques well known by those of ordinary skill in the art. It is further contemplated that the GGTase and FTase inhibitory compounds of the invention may be formulated in intraocular inserts or implantable devices.

The GGTase and FTase inhibitory compounds disclosed herein are preferably incorporated into topical ophthalmic formulations for delivery to the eye. The compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

GGTase and FTase inhibitory compounds are preferably formulated as topical ophthalmic suspensions or solutions, with a pH of about 4 to 8. The compounds are contained in the topical suspensions or solutions in amounts sufficient to lower IOP in patients experiencing elevated IOP and/or maintaining normal IOP levels in glaucoma patients. Such amounts are referred to herein as “an amount effective to control IOP,” or more simply “an effective amount.” The compounds will normally be contained in these formulations in an amount 0.01 to 5 percent by weight/volume (“w/v %”), but preferably in an amount of 0.25 to 2 w/v %. Thus, for topical presentation 1 to 2 drops of these formulations would be delivered to the surface of the eye 1 to 4 times per day, according to the discretion of a skilled clinician.

The GGTase and FTase inhibitory compounds can also be used in combination with other elevated IOP or glaucoma treatment agents, such as, but not limited to, rho kinase inhibitors, β-blockers, prostaglandin analogs, carbonic anhydrase inhibitors, α₂ agonists, miotics, and neuroprotectants.

### Determination f Biological Activity

In vitro Biological Activity Assays

The ability of certain compounds to inhibit GGTase and FTase may be evaluated in certain embodiments by in vitro assays, such as the in vitro prenyltransferase assays described by Burke et al., PNAS, Vol. 96:23:13062-13067, 1999 and Goossens et al., J. Pharm. Biomed. Analy., Vol. 37:417-422, 2005. Briefly, using the method of Goossens, experimental and control preparations comprising GGTase or FTase along with dansylated peptide substrates for either enzyme were made. Test compound is added to the experimental preparation, and the reaction is allowed to proceed. Following the reaction, the fluorescent response of each peptide is measured, with a decrease in measured fluorescence compared to control representing greater inhibitory activity for the test compound.

### In Vivo Biological Activity Testing

The ability of certain GGTase and FTase inhibitory compounds to safely inhibit the respective enzymes may be evaluated in certain embodiments by means of in vivo assays using New Zealand albino rabbits and/or Cynomolgus monkeys.

### Ocular Safety Evaluation in New Zealand Albino Rabbits

Both eyes of five New Zealand albino rabbits are topically dosed with one 30 μL aliquot of a test compound in a vehicle and five additional animals are dosed with vehicle alone. Animals are monitored continuously for 0.5 hr post-dose and then every 0.5 hours through 2 hours or until effects are no longer evident.

### Acute IOP Response in New Zealand Albino Rabbits

Intraocular pressure (IOP) is determined with a Mentor Classic 30 pneumatonometer after light corneal anesthesia with 0.1% proparacaine. Eyes are rinsed with one or two drops of saline after each measurement. After a baseline IOP measurement, test compound is instilled in one 30 μL aliquot to one or both eye of each animal or compound to one eye and vehicle to the contralateral eye. Subsequent IOP measurements are taken at 0.5, 1, 2, 3, 4, and 5 hours.

### Acute IOP Response in Cynomolgus Monkeys

Intraocular pressure (IOP) is determined with an Alcon pneumatonometer after light corneal anesthesia with 0.1% proparacaine as previously described (Sharif et al., J. Ocular Pharmacol. Ther., Vol. 17:305-317, 2001; May et al., J. Pharmacol. Exp. Ther., Vol. 306:301-309, 2003). Eyes are rinsed with one or two drops of saline after each measurement. After a baseline IOP measurement, test compound is instilled in one (300 μg) or two (600 μg) 30 μL aliquots to the selected eyes of nine cynomolgus monkeys. Vehicle is instilled in the selected eyes of six additional animals. Subsequent IOP measurements are taken at 1, 3, and 6 hours. Right eyes of all animals had undergone laser trabeculoplasty to induce ocular hypertension. All left eyes are normal and thus have normal IOP.

### Examples

The following examples are provided to illustrate certain embodiments of the invention, but should not be construed as implying any limitations to the claims. For example, the phrase “Prenyltransferase Inhibitor” in Example 4 means that the formulation described is believed to be suitable for any GGTase and FTase inhibitory compound disclosed herein.

### Example 1

### RNA Isolation and Quantitative RT-PCR

Total RNA was isolated from TM cells using Qiagen RNeasy 96 system according to the manufacturer's instructions (Qiagen).

Differential expression of CTGF and PAI-1 were verified by quantitative real-time RT-PCR (QRT-PCR) using an ABI Prism® 7700 Sequence Detection System (Applied Biosystems) essentially as previously described (Shepard et al., IOVS, 2001, Vol. 42:3173). Primers for CTGF amplification were designed using Primer Express software (Applied Biosystems) to anneal to adjacent exons of Genbank accession #NM_{—}001901.1 (CAGCTCTGACATTCTGATTCGAA (SEQ ID NO: 1), nts 1667-1689 and TGCCACAAGCTGTCCAGTCT (SEQ ID NO: 2), nts 1723-1742, with probe sequence 6FAM-AATCGACAGGATTCCGATTCCTGAACAGTG-TAMRA (SEQ ID NO: 3)) and generate a 76-bp amplicon. Primers for PAI-1 amplification were purchased from ABI (Hs00167155 ml) and correspond to Genbank accession #NM_{—}000602.1. Amplification of CTGF or PAI-1 was normalized to 18S ribosomal RNA expression using primers designed to the 18S rRNA gene (GenBank accession #X03205 GTCCCTGCCCTTTGTACACAC (SEQ ID NO: 4), nts 1680-1700 and CGATCCGAGGGCCTCACTA (SEQ ID NO: 5), nts 1730-1749, with probe sequence 6FAM-CTGCAAGCATATAATACA-MGBNFQ (SEQ ID NO: 6)) which generates a 69-bp amplicon. CTGF or PAI-1 QRT-PCR was performed in multiplex with 18S primer/probe sets in a 50 ul final volume consisting of 40 nM 18S or 900 nM CTGF or PAI-1 primers; 100 nM 18S probe or 100 nM CTGF or 250nM PAI-1 probe; 5 ul RNA; 1× Multiscribe and RNase Inhibitor Mix (ABI); and 1× TagMan® Universal Mix (ABI). Thermal cycling conditions consisted of 48° C., 30 min, 95° C. 10 min followed by 40 cycles at 95° C., 15 sec, 60° C., 1 min. Data analysis was performed with SDS software version 1.9.1 (Applied Biosystems) and MS Excel 2002 (Microsoft). Quantification of relative RNA concentrations was done using the delta delta Ct method as described in PE Biosystems User Bulletin #2. Levels of amplified products were expressed as mean ±SEM of quadruplicate QRT-PCR assays. Data analysis was performed with SDS software version 1.9.1 (Applied Biosystems) and MS Excel 97 (Microsoft).

### Example 2

### Inhibition of TGFβ-Stimulated CTGF and PAI-1 Gene Expression

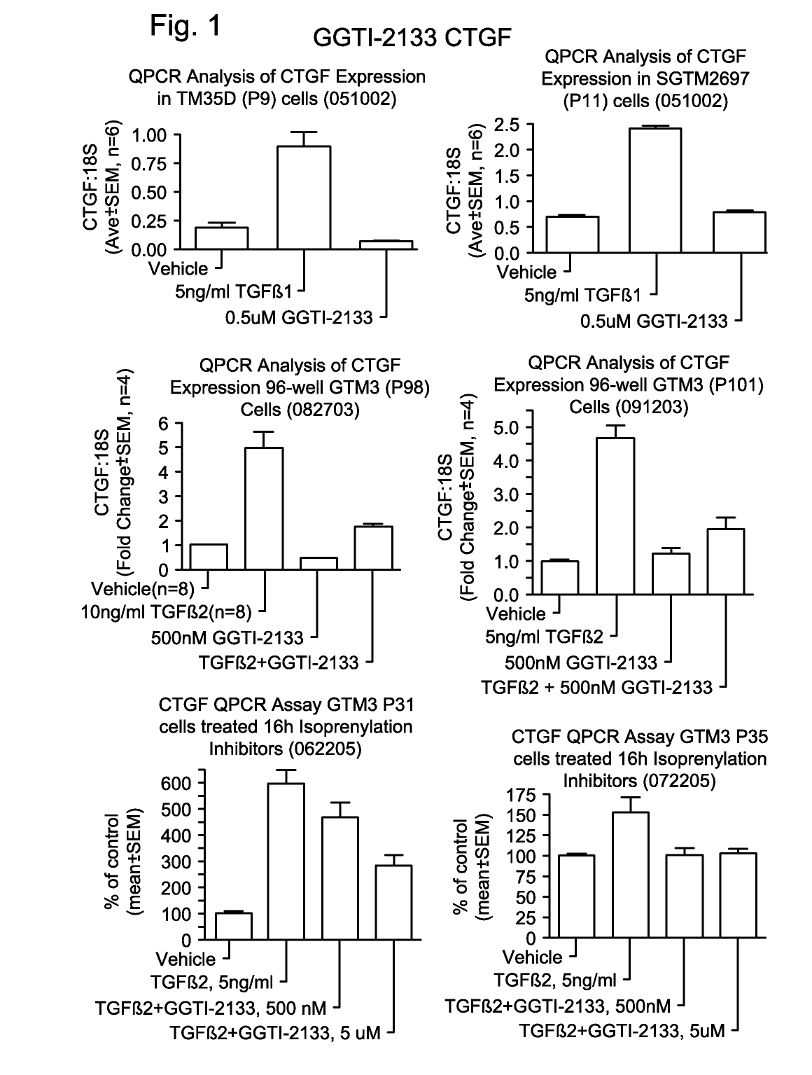

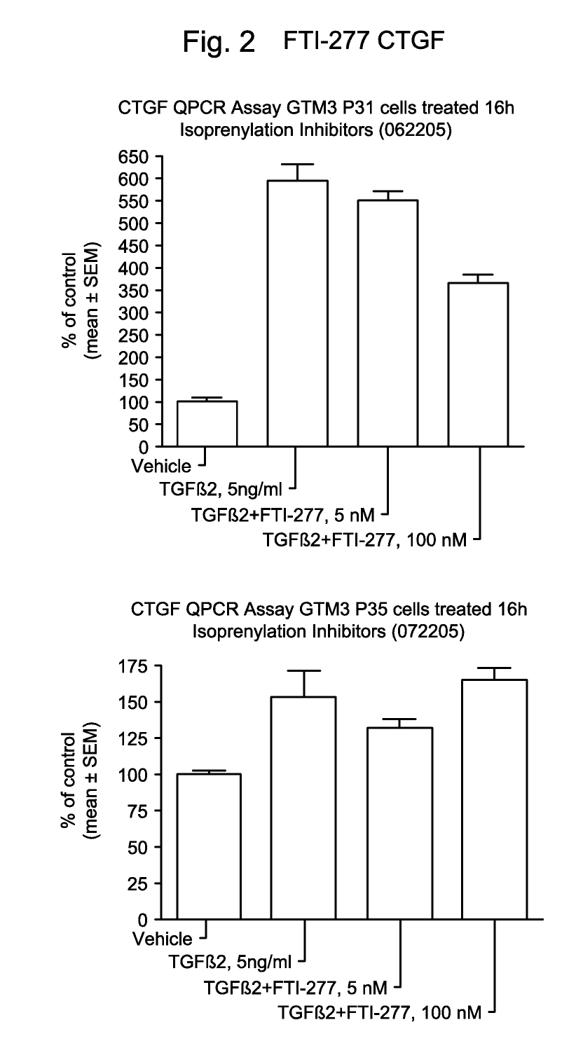

In this example, the effectiveness of GGTase and FTase inhibitors on CTGF gene expression in cultured human trabecular meshwork cells was studied. The results are summarized in FIGS. 1 and 2. In this experiment, the CTGF/18S cDNA levels were measured and compared by QRT-PCR according to the protocol of Example 1.

As can be seen from the summary of the results in FIG. 1, a GGTase inhibitor, GGTI-2133, was tested to determine its effect on CTGF levels in various

TM cell cultures. As shown in FIG. 1, when TGFβ2 was present in the vehicle, the measured CTGF levels were elevated compared to vehicle alone. In cell cultures treated with both CTGF and GGTI-2133, measured CTGF levels were lower than with vehicle alone, and had dramatically reduced CTGF levels compared to the TGFβ2-treated cells.

The results shown in FIG. 2 illustrate that the FTase FTI-277 also produces a drop in measured CTGF levels when cell lines treated with TGFβ2 alone are compared to cell lines treated with both TGFβ2 and FTI-277.

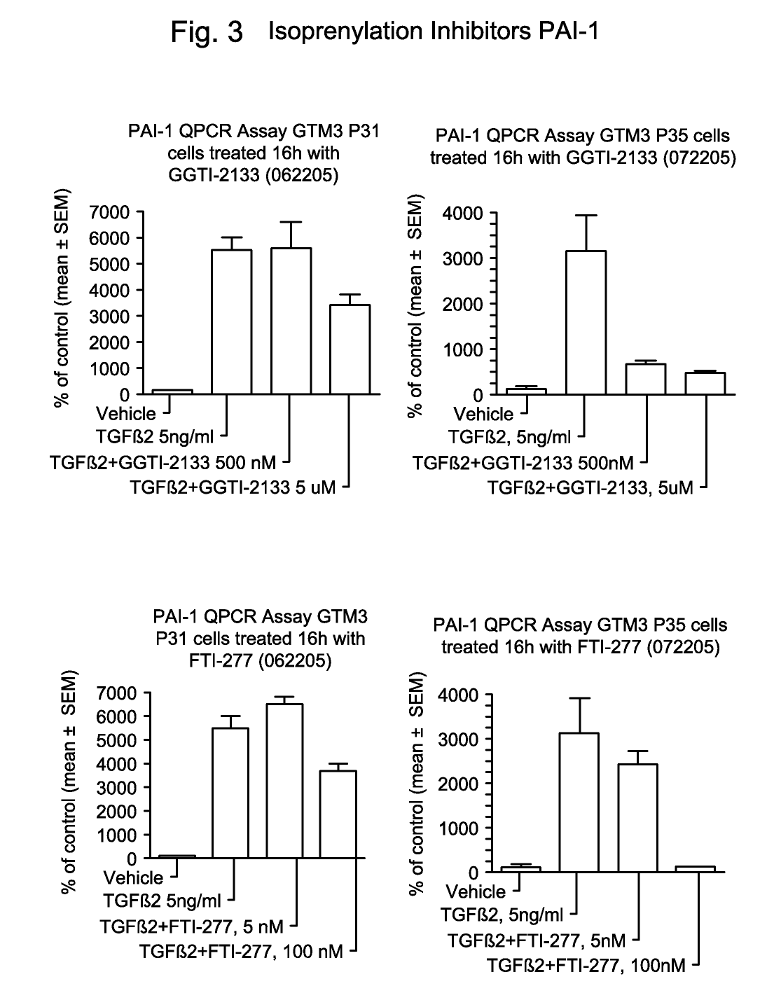

FIG. 3 illustrates that both GGTI-2133 and FTI-277 were able to produce drops in measured PAI-1 when cell lines treated with TGFβ2 alone are compared to cell lines treated with both TGFβ2 and GGTI-2133 or FTI-277.

### Example 3

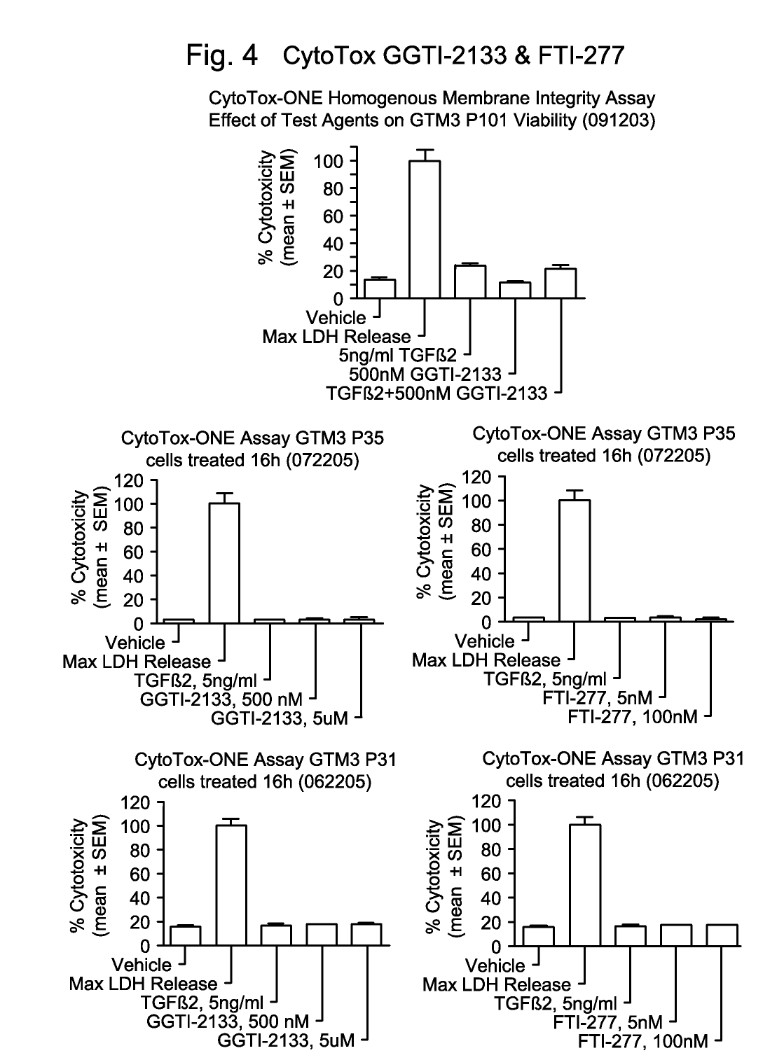

FIG. 4 shows graphs presenting cytotoxicity effects of GGTI-2133 and FTI-277 using the CytoTox-ONE Homogenous Membrane Integrity Assay (Promega) which measures lactate dehydrogenase (LDH) release into culture media after treatment with test compounds. Both compounds, at all concentrations tested, had similar LDH release measurements to vehicle alone measurements. Both compounds thus appear to have relatively low cytotoxicity.

Example 4

| Ingredients | Concentration (w/v %) |
|---|---|
| Prenyltransferase Inhibitor Compound | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

Example 5

| Ingredients | Concentration (w/v %) |
|---|---|
| Prenyltransferase Inhibitor Compound | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

Example 6

| Ingredients | Concentration (w/v %) |
|---|---|
| Prenyltransferase Inhibitor Compound | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

Example 7

| Ingredients | Concentration (w/v %) |
|---|---|
| Prenyltransferase Inhibitor Compound | 0.01-2% |
| White petrolatum and mineral oil and | Ointment consistency |
| lanolin | |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

1. A method of treating glaucoma or elevated intraocular pressure associated with elevated levels of connective tissue growth factor (CTGF) and/or plasminogen activator inhibitor-1 (PAI-1) production comprising:
administering a pharmaceutically effective amount of a composition comprising at least one prenyltransferase inhibitor, thereby inhibiting production of CTGF and/or PAI-1 and treating said glaucoma or elevated intraocular pressure.

2. The method of claim 1 wherein said at least one prenyltransferase inhibitor is a geranylgeranyltransferase inhibitor or a farnesyltransferase inhibitor.

3. The method of claim 1 wherein said administering comprises administering a composition comprising at least one geranylgeranyltransferase inhibitor and at least one farnesyltransferase inhibitor.

4. The method of claim 1 wherein said composition further comprises a compound selected from the group consisting of:
ophthalmologically acceptable preservatives, surfactants, viscosity enhancers, penetration enhancers, gelling agents, hydrophobic bases, vehicles, buffers, sodium chloride, and water.

5. The method of claim 1, further comprising administering, either as part of said composition or as a separate administration, a compound selected from the group consisting of:
β-blockers, prostaglandin analogs, carbonic anhydrase inhibitors, α₂ agonists, miotics, neuroprotectants, and any combination thereof

6. The method of claim 1 wherein said composition comprises from about 0.01 percent weight/volume to about 5 percent weight/volume of said at least one prenyltransferase inhibitor.

7. The method of claim 1 wherein said composition comprises from about 0.25 percent weight/volume to about 2 percent weight/volume of said prenyltransferase inhibitor.

8. A composition for the treatment of elevated intraocular pressure and glaucoma associated with elevated levels of connective tissue growth factor (CTGF) and/or plasminogen activator inhibitor-1 (PAI-1) production comprising:
a pharmaceutically effective amount of a prenyltransferase inhibitor.

9. The composition of claim 8 wherein said prenyltransferase inhibitor is a geranylgeranyltransferase inhibitor or a farnesyltransferase inhibitor.

10. The composition of claim 8, further comprising a compound selected from the group consisting of:
ophthalmologically acceptable preservatives, surfactants, viscosity enhancers, penetration enhancers, gelling agents, hydrophobic bases, vehicles, buffers, sodium chloride, and water.

11. The composition of claim 8 wherein said composition comprises from about 0.01 percent weight/volume to about 5 percent weight/volume of said prenyltransferase inhibitor.

12. The composition of claim 8 wherein said composition comprises from about 0.25 percent weight/volume to about 2 percent weight/volume of said prenyltransferase inhibitor.

13. The composition of claim 8 wherein said composition further comprises a compound selected from the group consisting of:
β-blockers, prostaglandin analogs, carbonic anhydrase inhibitors, α₂ agonists, miotics, neuroprotectants, rho kinase inhibitors, and any combination thereof.

14. The composition of claim 8 wherein said prenyltransferase inhibitor is selected from the group consisting of:
GGTI-286, GGTI-287, GGTI-297, GGTI-298, GGTI-2133, GGTI-2147, FTI-276, FTI-277, FTI-2148, FTI-2153, R115777, combinations thereof, and pharmaceutically acceptable salts thereof.

15. A method of treating glaucoma or elevated intraocular pressure associated with elevated levels of connective tissue growth factor (CTGF) and/or plasminogen activator inhibitor-1 (PAI-1) production, which comprises administering to a human or other mammal a therapeutically effective amount of a compound selected from the group consisting of:
GGTI-286, GGTI-287, GGTI-297, GGTI-298, GGTI-2133, GGTI-2147, FTI-276, FTI-277, FTI-2148, FTI-2153, R115777, combinations thereof, and pharmaceutically acceptable salts thereof.
